(19) United States
(12) Patent Application Publication
(10) Pub. No.: US 2025/0006302 A1
(43) Pub. Date: Jan. 2, 2025

(54) METHOD FOR PREDICTING RELATIONSHIP BETWEEN BACTERIOPHAGE AND BACTERIAL INFECTION

(71) Applicant: BGI SHENZHEN, Shenzhen (CN)

(72) Inventors: Min LI, Shenzhen (CN); Nianji ZHAN, Shenzhen (CN); Minfeng XIAO, Shenzhen (CN); Yong BAI, Shenzhen (CN); Xin JIN, Shenzhen (CN); Wenchen SONG, Shenzhen (CN); Hailin PAN, Shenzhen (CN); Tongda ZHANG, Shenzhen (CN)

(73) Assignee: BGI SHENZHEN, Shenzhen, GD (CN)

(21) Appl. No.: 18/705,956

(22) PCT Filed: Nov. 3, 2021
(86) PCT No.: PCT/CN2021/128492

§ 371 Date: Apr. 29, 2024

(51) Int. Cl. G16B 20/20; C12Q 1/70; G06N 20/00

(52) U.S. Cl. CPC G16B 20/20; C12Q 1/70; G06N 20/00

(57) ABSTRACT

A method for predicting the relationship between a bacteriophage and a bacterial infection, comprising a learning model for a bacteriophage-host infection relationship and a construction method therefor. The method for constructing a learning model for a bacteriophage-host infection relationship comprises: training a learning model by using data of a training set of bacteriophages and bacterial strains, the data of the training set of bacteriophages and bacterial strains comprising genome data and phenotypic assay data. A method for using the learning model for a bacteriophage-host infection relationship to predict the bacteriophage that will infect bacteria and a method for predicting the bacteria infected by a bacteriophage, and a computer-executable medium comprising a computer program for performing the methods.

### FIELD OF THE INVENTION

The present invention belongs to the field of biotechnology, and more specifically, the present invention provides a method for predicting infection relationship between bacteriophages and bacteria.

### BACKGROUND OF THE INVENTION

In recent years, with the spread of antibiotic-resistant bacteria worldwide, the problem of antibiotic-resistant infection has become increasingly serious, which has prompted some scientists to devote themselves to the research of bacteriophage therapy. However, due to the high specificity of bacteriophages, the host profiles of different strains of pathogens in same genus are quite different. It is still a major challenge in clinical application to quickly and accurately find the corresponding and available bacteriophages when the target pathogens are known. At present, only traditional experimental methods (such as spot assay, microfluidic-PCR and PhageFish) can be used to confirm whether there is infection relationship between bacteriophages and bacteria. However, experimental identification would take at least several days, depending on the number of bacteriophage hosts. This has largely limited the clinical application of bacteriophage therapy. Meanwhile, the volume of current bacteriophage resource libraries and databases is particularly scarce. NCBI GeneBank, EMBL-EBI and Phantom, the most famous large-scale databases worldwide, only have genomic information of about 10,000 bacteriophages, and the corresponding host information is very scarce and unclear. This brings inconvenience to bacteriophage related research, modification and therapy.

Therefore, to solve the drawbacks of bacteriophage therapy and promote the use of bacteriophage in the treatment of bacteria-related diseases, the problems to be encountered include quickly completing the accurate matching of bacteriophages against target strains, and how to scientifically select bacteriophages and configure bacteriophage combinations when there are multiple strains of bacteriophages that can lyse a specific host.

When bioinformatics, genomics and next-generation sequencing technology were not developed and popularized, bacteriophage mining initially only utilized experimental method of host bacteria targeted screening of natural lytic bacteriophages. The experimental method has a series of drawbacks, such as low efficiency, consuming time, poor targeting, slow reaction speed, high cost, and accidental and random isolation and screening. The technical solution is as follows: host bacteria and candidate bacteriophage samples are co-cultured, the lysis phenomenon is observed after amplification, and the bacteriophage samples that could infect the host bacteria are further obtained. Further, the bacteriophage samples would be used to clinically kill pathogens under the condition that the bacteriophages are not completely sequenced and understood, that is, whether they contained virulence genes is unknown.

At present, some have proposed to apply computational methods to find host bacteria corresponding to bacteriophages, such as the computational biology-based method and matched bioinformatics tools thereof HostPhinder proposed by Larsen et al. (Villarroel J, Kleinheinz K A, Jurtz V I, et al. HostPhinder: a phage host prediction tool[J]. Viruses, 2016, 8(5): 116). The tools can predict infection relationship between a candidate bacteriophage and a potential host by comparing the similarity between the genome of the bacteriophage and that of the known bacteriophage of the potential host. Edwards et al. have also proposed a method for comparing sequence similarity based on BLAST to further determine the bacteriophage-host relationship (Edwards R A, McNair K, Faust K, et al. Computational approaches to predict bacteriophage-host relationships[J]. FEMS microbiology reviews, 2016, 40(2): 258-272). Most of these methods rely entirely on sequence similarity as a prerequisite, which makes it difficult to make prediction in the absence of known genetically related bacteriophage-bacterium pairing relationship.

Machine learning strategy is expected to break through alignment methods based on sequence similarity and reduce the dependence on the sequences contained in known data. Leite et al. (Leite D M C, Brochet X, Resch G, et al. Computational prediction of inter-species relationships through omics data analysis and machine learning[J]. BMC bioinformatics, 2018, 19(14): 151-159) have recently proposed a predictive model based on machine learning to determine the relationship between bacteriophages and candidate bacterial hosts. This method takes the protein interaction relationship between bacteriophages and bacteria as the starting point, extracts the interactions among protein domains and primary structure information of protein as input features to train a variety of machine learning classification models, and finally, obtains the optimal result on the artificial neural network (ANN), with the classification accuracy of 90.4%. However, this method has three shortcomings. Firstly, in the construction of datasets, the data sources lack experimental support, and data construction is not scientific and systematic enough. In this study, only genomic data of one-to-one bacteriophages and host bacteria with marked infection relationship in the network database are as positive sample data. The construction of training dataset is based on interaction among all proteins, but there is a lot of redundancy in the dataset because only a small number of proteins are involved in bacteriophage-host interaction, and there is nucleic acid-protein interaction during the bacteriophage replication and translation, leading to incomplete dataset. Researchers use the data other than bacteriophage-bacterium pairs with interaction published in the online databases as negative sample data. This proposing lacks any experimental data and is extremely one-sided. When selecting input features, researchers simply take the mean and variance of all feature pairs as input features, which is not scientific and systematic. Secondly, in terms of prediction accuracy, this method can only predict infection relationship at the strain level, which cannot meet the actual practice application. The host specificity of bacteriophage is extremely high, and they are usually only able to infect some strains in the same species. Thirdly, the prediction results can only provide qualitative prediction, i.e. YES or NO, but cannot quantitatively predict the ability of bacteriophage in lysing host and solve the problem of bacteriophage selection when there are multiple strains of bacteriophages that can lyse the same host simultaneously.

Therefore, all of current predictive mining methods are limited by the lack of existing bacteriophage genome and experimental data. For both of the method being based on the alignment of bacteriophage genome similarity and the method using machine learning methods to train predictive models, they have relatively limitation because of the complex species diversity of bacteriophages and the unknown and huge number of bacteriophages in nature. Further, due to the lack of means for follow-up verification of prediction results, the occurrence of false positive and false negative cannot be selectively avoided. The prediction results of the existing machine learning methods can only provide qualitative prediction, i.e. YES or NO, and cannot quantitatively predict the ability of bacteriophage in lysing host and solve the problem of bacteriophage selection when there are multiple strains of bacteriophages that can lyse specific host simultaneously.

### SUMMARY OF THE INVENTION

Focusing on the defects of prior art, the present invention provides a model, which is based on phylogenetic analysis combined with deep learning, to learn the genotype-phenotype corresponding relationship based on the whole genomic data of bacteriophages and bacteria, and complete the prediction of bacteriophage-host infection relationship with accuracy to strain level.

Therefore, in a first aspect, the present invention provides a method for constructing a learning model for a bacteriophage-host infection relationship, the method comprising:
training a learning model by using data of bacteriophages and bacterial strains in the training set, the data of bacteriophages and bacterial strains in the training set comprising genomic data and phenotypic data from experimental assay.

In one embodiment, the phenotypic data from experimental assay are the quantitative infection data of bacteriophages and bacterial strains, such as bacteriophage-bacterium infection score.

In one embodiment, the bacteriophage-bacterium infection score is calculated as follows:
1) circularity is calculated by:

circularity=4×π×area of plaque+(perimeter of plaque×perimeter of plaque) (1),

preferably, the plaques with circularity of less than a threshold (e.g., 0.1) are removed;
2) transmissivity of plaque is calculated by:

transmissivity of plaque=brightness value of plaque/brightness value of background (2),
preferably, the plaques with transmissivity of greater than a threshold (e.g., 0.995) are removed;
3) the infection score is calculated by:

infection fraction=circularity+20×(1−transmissivity of plaque) (3).

In one embodiment, the data of bacteriophages and bacterial strains in the training set comprise the data of bacteriophages and bacterial strains without infection relationship, for example, the infection score is less than 1.5.

In one embodiment, in the bacteriophages and bacterial strains in the training set, there is the combination of two or more bacteriophages and one bacterial strain; preferably, the two or more bacteriophages are known to infect the one bacterial strain.

In one embodiment, the genomic data comprise genomic data of the bacteriophages and bacterial strains, such as genomic sequences; preferably, the genomic data are SNP datasets.

In one embodiment, the SNP datasets comprise the loci where there is difference in the base type on the whole genome or part of genome when individual genome is aligned with reference genome, wherein the loci whose similarity of proportion of base type with the reference genome is greater than a threshold (e.g., 0.9), and invalid loci are deleted.

In one embodiment, the data of bacteriophages and bacterial strains in the training set are eigenvector matrix comprising eigenvector of the bacteriophages and eigenvector of the bacteria.

In one embodiment, the bacterial strains are from the same genus; preferably, the bacterial strains in the training set are from same species.

In one embodiment, the bacteriophages in the training set are the bacteriophages from same phylogenetic clade cluster.

In one embodiment, the reference sequence of the bacteriophages is the ancestral sequence in each clade.

In one embodiment, the reference sequence of the bacteria is the reference genome sequence of the bacteria recorded in the NCBI database.

In one embodiment, one or more bacterial strains in same species are infected by one or more bacteriophages in the phylogenetic clade cluster.

In one embodiment, the phylogenetic clade cluster is divided based on genomic data of the bacteriophages, e.g., by cluster analysis.

In one embodiment, data of the bacteriophages and bacterial strains are merged to form an eigenvector matrix as the input of the learning model; for example, the eigenvector of the bacteriophages is repeated multiple times until its dimension is consistent with that of the eigenvector of the bacterial strains, and the repeated eigenvector of the bacteriophages and the eigenvector of the bacterial strains constitute the eigenvector matrix.

In one embodiment, the learning model is based on neural network framework.

In one embodiment, the learning model comprises a pre-trained model and a master model.

In one embodiment, the pre-trained model adopts a standard symmetric autoencoder model, comprising an encoder, a bottleneck layer and a decoder.

In one embodiment, the master model adopts the encoder in the pre-trained model and two layers of full connection.

In one embodiment, the models trained by multiple different phylogenetic clades are combined to form model combinations, and the model combinations constitute the learning model.

In a second aspect, the present invention provides a trained learning model obtained by the method of the first aspect of the present invention.

In a third aspect, the present invention provides a method for predicting bacteriophages that are capable of infecting a bacterium, the method comprising:
the genomic data of the bacterium to be tested and each of candidate bacteriophages are input into the learning model of the second aspect of the present invention, to predict the bacteriophages or bacteriophage combinations capable of infecting the bacterium to be tested.

In one embodiment, the candidate bacteriophages comprise the bacteriophages used to train the learning model.

In a fourth aspect, the present invention provides a method for predicting bacteria infected by a bacteriophage, the method comprising:
the genomic data of the bacteriophage to be tested and each of candidate bacteria are input into the learning model of the second aspect of the present invention, to predict the bacteria that the bacteriophage to be tested are capable of infecting.

In one embodiment, the candidate bacteria comprise the bacteria used to train the learning model.

In a fifth aspect, the present invention provides a method for predicting the paired infection relationship between bacteria and bacteriophages, the method comprising:
the genomic data of the bacteriophages and bacteria to be tested are pairwise input into the learning model of the second aspect of the present invention, to predict whether there is infection relationship between the bacteriophages to be tested and the bacteria to be tested, and the infection of the bacteriophages to be tested to the bacteria to be tested.

In a sixth aspect, the present invention provides a computer execution medium comprising a computer program, wherein the computer program is used to execute the method steps of the third aspect to fifth aspect of the present invention.

The method of the present invention has realized that bacteriophage genome sequence or bacterial genome or bacteriophage-bacterium combination are input into packaged software in clinical therapy to predict and output infected bacterial strains or infective bacteriophage strains or whether there is infection relationship between bacteriophages and bacteria, corresponding infection score, infection possibility and the optimal combination scheme in a short time. This provides rational guidance for clinical application of bacteriophage therapy.

### BRIEF DESCRIPTION OF THE DRAWINGS

FIG. 1 shows an example of phylogenetic analysis of bacteriophages, wherein the clustering is performed starting from the closest clade from the middle center.

FIG. 2 shows the genomic sequence processing and data merging processing steps of bacteria and bacteriophages, including alignment with the reference sequence, filtering SNP loci information, one-hot encoding locus bases, and merging data.

FIG. 3 shows the internal design of a deep neural network. The top half of the figure represents the pre-trained model, including two steps, i.e., encoding region and decoding region, and the encoding and decoding regions contain two layers of CBR module and DeCBR module, respectively, which are connected by the bottleneck layer; the bottom half of the figure represents the master model, which retains the encoding region of the former part, and is followed by two fully connected layers as the classification layer to output the classification results.

FIG. 4 shows prediction results of the model.

FIG. 5 shows infection score matrix of bacteriophages and bacteria obtained by method and system for quantitative analysis of high-throughput bacteriophage antimicrobial phenotype.

### DETAILED DESCRIPTION OF THE EMBODIMENTS

The technical solution of the present invention takes Klebsiella pneumoniae bacteriophage as an example to introduce model construction, testing and verification based on Klebsiella pneumoniae bacteriophage. It should be understood that the overall route and framework of the technical solution of the present invention are suitable to predict a wide range of bacteriophage-bacterium interaction. The technical route of the present invention includes data acquisition, data preprocessing, model prediction, results obtaining and analysis. The method of the present invention is introduced in the following four aspects: processing of raw data, construction method of model, training and prediction of model and result analysis.

### 1. Data Acquisition and Construction of Training Set

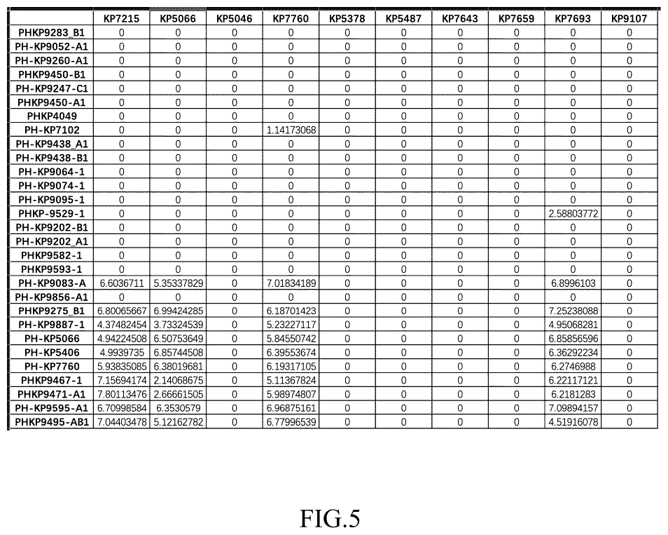

The first step is to obtain the quantitative infection dataset, utilizing the platform in the patent application of Method and System for Quantitative Analysis of High-throughput Bacteriophage Antimicrobial Phenotype (Patent Application No. PCT/CN2020/131879, which is incorporated herein in its entirety) to perform one-to-one paired high-throughput host profile experiment for 129 strains of Klebsiella pneumoniae bacteriophages and 101 strains of different Klebsiella pneumoniae respectively, to obtain the host profile information represented by one-to-one infection score of all bacteriophages and bacteria. Here, the experiment results of host profile include both the positive sample phenotypic data (infection score) of the bacteriophage-bacterium pairs that can produce bacteriophage plaques, i.e., have infection relationship, and the negative sample phenotypic data (infection score) of the bacteriophage-bacterium pairs, which cannot produce obvious bacteriophage plaques and have no infection relationship. The quantitative infection dataset consists of a matrix of bacteriophage-bacterium infection scores, expressed as:

$$[ \begin{matrix}ID & … & Bacteria - n \\ ⋮ & ⋱ & ⋮ \\ Phage - m & … & 3.7568\end{matrix} ] ,$$

wherein the first row of the matrix represents the bacterial number, the first column represents the bacteriophage number, and the orthogonal corresponding scores of the two are the infection scores obtained from method and system for quantitative analysis of high-throughput bacteriophage antimicrobial phenotype, where 3.7568 is an example infection score of Phage-m and Bacteria-n. FIG. 5 shows data format of the actual matrix.

The experimental steps of high-throughput host profile experiment by Method and System for Quantitative Analysis of High-throughput Bacteriophage Antimicrobial Phenotype (Patent Application No. PCT/CN2020/131879) are briefed as follows: PFU is determined for the bacteriophages, and after all of the bacteriophage solution to be tested is diluted to 108 PFU, addition of bacteriophage and bacterial solution, timed photographing, converting images to data and other operations are performed according to the instruction of Method and System for Quantitative Analysis of High-throughput Bacteriophage Antimicrobial Phenotype, to finally obtain the matrix of infection scores. Infection score is a numerical indicator to comprehensively evaluate bacteriophage infection ability by using image recognition algorithm to identify the photographed plaque images, and comprehensively calculating area of plaque, halo aperture size and transmissivity of plaque. An exemplary infection score can be calculated as follows:

First, determining shape parameter of plaque: because most of plaques are round or oval, and shapes of plaques or impurities with lower contrast are generally irregular, the inventor takes circularity of plaque as one of the confident parameters of plaque, and uses formula (1) to calculate circularity of plaque:

$$circularity = 4 \times π \times area \div ( perimeter \times perimeter ) , \qquad ( 1 )$$

wherein the area and perimeter are the area and perimeter of a plaque respectively, the perimeter is the length of the plaque line (unit: pixel), and the area is the area of the plaque (unit: pixel). A higher circularity indicates a rounder target region with a value of 0-1. When the shape parameter is less than 0.1, the target region is regarded as impurity.

The transmissivity parameter of plaque: because the brightness of plaque is generally darker (relative to the surrounding), the inventor uses the transmissivity as one of the confident parameters of plaque. The plaque and its surrounding halo are firstly detected, then the upper, lower, left and right ranges of the plaque are measured, the plaque (inner circle region) is labeled to expand to the left, right, up and down respectively as background preselection region, and then all of the plaques and halos in the calculated region (including other plaques and halos that might be possibly trapped) are removed. The median of brightness values of pixels (converted to gray values) in the defined region is taken as the brightness value of plaque, and the median of brightness values of pixels (converted to gray values) outside the defined region is taken as the brightness value of background brightness. The transmissivity of plaque is calculated as follows,

$$transmissivity of plaque = brightness value of plaque / brightness value of background . \qquad ( 2 )$$

In general, the transmissivity is 0.5-1, and a lower transmissivity indicates a darker target region and a greater possibility of plaque. Preferably, plaque with transmissivity of greater than 0.995 can be considered as impurity and needs to be removed.

Calculation of infection score: because the range of transmissivity is 0.5-1, a lower transmissivity indicates a greater probability that the target is a plaque, and a transmissivity has stronger guiding significance for the determination of plaque. Thus, the formula for calculating infection score of the final target region is:

$$infection score = circularity + 20 \times ( 1 - transmissivity of plaque ) . \qquad ( 3 )$$

The level of infection score can relatively represent the strength of bacteriophage infection ability. In the test by the inventor, it is found that all of the images with infection score of greater than 1 are experimentally verified as positive plaques, the images with infection score of (0,1) are experimentally verified to have 15% probability of being positive plaques, and all of the images with infection score of less than or equal to 0 are experimentally verified as negative plaques.

Since the phenotype of bacteriophage, i.e., the characterization of infection performance, is initially not clear, it is difficult to identify the specific functional bacteriophage strains if multiple bacteriophages are blindly mixed. Starting from a single strain, the infection performance of each strain of bacteriophage against different bacteria can be determined, and the synergistic or antagonistic effect between bacteriophage combinations is explored by combinations of pairings (two strains of bacteriophages corresponding to a strain of bacterium) or combinations of more bacteriophages (multiple strains of bacteriophages corresponding to a strain of bacterium). Compared with the combinations of pairings or more bacteriophages, a single bacteriophage-host infection system is clearer and easier to control. To ensure the authenticity and high stability of the training data input into model, the construction of training data for the model, the construction of the model and other processes are described in detail with a single strain of bacteriophage-host infection system as an example. In practical application, this method can be applied in a single strain of bacteriophage-host prediction and utilize simultaneously actual infection data or predicted infection data to popularize the application of multiple bacteriophages-hosts system.

The second step is the preparation of genomic data. The inventor collects genomic sequences of the corresponding bacteriophages and bacteria. The data sources include the whole genome data by sequencing the collected samples or downloading genomic data of samples directly from the Internet if they have been published publicly on the Internet.

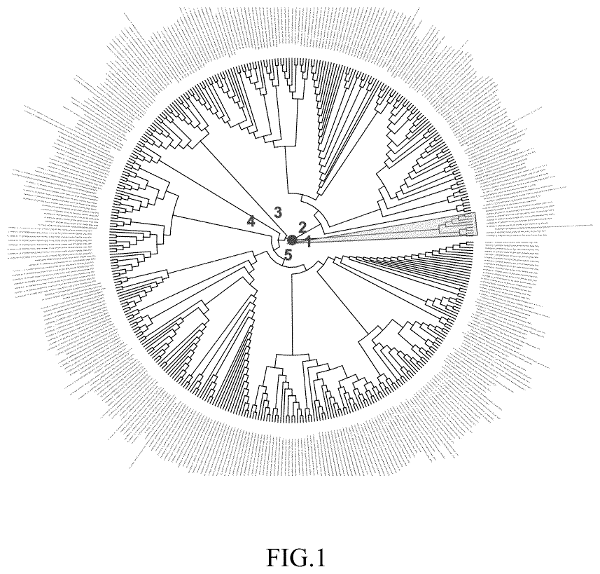

The third step is the construction of bacteriophage evolutionary tree dataset. Sequence global alignment is performed for all bacteriophage genomes to obtain the post-aligned format files (with suffix of such as .maff, .fasta and .rxml), which are input into the authoritative phylogenetic tool MEGA for phylogenetic analysis and output of binary tree files, so as to obtain phylogenetic information among bacteriophages. Phylogenetic information mainly includes homology and similarity of sequences, phylogenetic relationship of each bacteriophage, and phylogenetic cluster module where each bacteriophage is located. The strategy of this example is to construct a model for each of different phylogenetic clade clusters to achieve prediction with high accuracy in the strain level, and all models are integrated and packaged for a wide range of clinical applications. FIG. 1 shows an example of phylogenetic analysis of bacteriophages, wherein the clustering is performed starting from the closest clade from the middle center. As shown in the figure, it can be clustered into 5 clusters from the phylogenetic analysis. With the first cluster as an example, shaded part is the sample information contained in the cluster.

According to the above three steps, bacteriophage sequence set Pseq composed of j bacteriophage samples and bacterial sequence set Bseq composed of k bacterial samples are obtained, both of which constitute the infection score dataset Xᵢ of j*k groups, wherein Xᵢ˜{P_{seq}_{ⱼ}ⁱ,B_{seq}_{ₖ}ⁱ}, i,j,k∈Z₀⁺,Z₀⁺ is a set of positive integers containing 0. Feature set is constructed to obtain Single Nucleotide Polymorphism (SNP) dataset by using genomic phylogenetic analysis and SNP at each locus of the whole genome. The SNP dataset includes loci where there is difference in the base type on the whole genome or part of genome when individual genome is aligned with the reference genome. As shown in FIG. 1, bacteriophage ancestor sequence (the earliest appearing in the phylogenetic relationship) in each clade is selected by bacteriophage phylogenetic analysis as the reference sequence P_Ref, and other sequences in the same clade are aligned with the reference sequence. The differential polymorphism of each nucleic acid locus is output as bacteriophage feature P_snpⱼⁱ=[s₀ⁱ, s₁ⁱ, s₂ⁱ, . . . , sₙⁱ]ⱼ, s∈{A, T, C, G}, wherein n is the locus location, and reference sequence feature of bacteriophage is P_Rsnpⱼⁱ=[s₀ⁱ, s₁ⁱ, s₂ⁱ, . . . , sₙⁱ]ⱼ, s∈{A, T, C, G}, wherein n is the locus location. All of bacteria are aligned with the reference sequence (B_ref) of Klebsiella pneumoniae genus in NCBI database to output the corresponding differential polymorphism of specific nucleic acid loci as bacterial feature B_snpₖⁱ[s₀ⁱ, s₁ⁱ, s₂ⁱ, . . . , sₙⁱ]ₖ, s∈{A, T, C, G}, wherein n is the locus location, and reference sequence feature of bacterium is B_Rsnpₖⁱ[s₀ⁱ, s₁ⁱ, s₂ⁱ, . . . , sₙⁱ]ₖ, s∈{A, T, C, G}, wherein n is the locus location. The SNP set of bacteriophage reference sequence and bacterial reference sequence is X_Rsnpᵢ˜{P_Rsnpⱼⁱ, B_Rsnpₖⁱ}. For other bacteria, B_refⁱ represents reference sequence of the bacterium, such as the bacterial sequence recorded in NCBI database. Next, SNP set X_snpᵢ˜{P_snpⱼⁱ, B_snpₖⁱ} of the sequences except the reference sequence is filtered to further reduce the data size. It is supposed that bacteriophage set of the i^{th} clade is taken, at a certain locus n:

$$SNP\_Ratio = {( \frac{{\sum }_{0}^{j} ( {{P}_{snp}}_{n}^{j} ∼ {P\_Rsnp}_{n}^{i} )}{j} )}^{i}$$

is the similarity proportion at the same SNP locus between base type of each individual and base of the reference genome. A threshold filterₛᵤₚ (e.g., default 0.9) is set, if it is greater than the threshold, the SNP locus is deleted from X_snp_{N}.

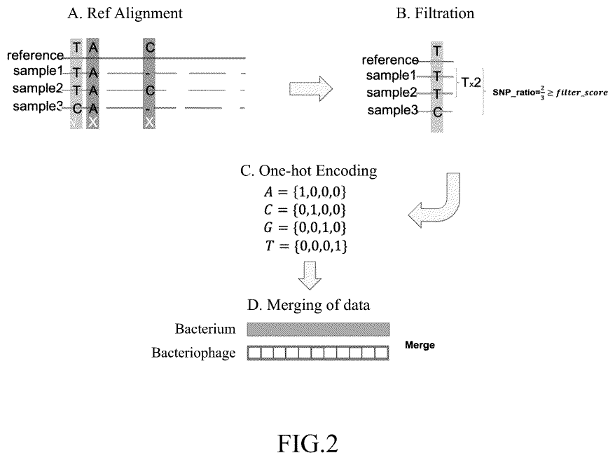

FIG. 2 shows the one-to-one genomic data processing and data merging processing of bacteria and bacteriophages, which is also suitable for the application of bacteriophage combinations and bacteria. According to FIG. 2, sequences of each bacteriophage and bacterium are aligned with reference sequences, and filtering SNP locus information, one-hot encoding locus base, and merging data are performed. In Ref alignment of A, for bacteriophages: a cluster of bacteriophages is firstly selected, in which the bacteriophage with the longest sequence is as the reference bacteriophage, the fasta sequences of these bacteriophages are used to generate the post-aligned maf files by sequence alignment software lastz, the maf files are analyzed to obtain and delete the loci with the similarity of base type proportion with reference genome of greater than 0.9 (see the above SNP_Ratio calculation formula), wherein the loci containing “N” or “-” and blank loci (these loci can be called invalid loci) are also deleted, and the SNP (differential polymorphism of nucleic acid loci) feature of bacteriophage is obtained; for bacteria: similarly, all bacterial data are aligned with a bacterial reference sequence to generate and analyze the maf files, and duplicated loci, loci containing “N” or “-” and blank loci (these loci can be called invalid loci) are deleted to obtain SNP feature of bacteria; B. filtration: each locus of the obtained bacteriophage and bacterial SNP features (equivalent to the reduced fasta sequences) is scanned and if the similarity proportion at the same locus between base type and base of the reference genome is greater than a threshold (e.g., default 0.9), the locus is deleted to obtain filtered SNP feature respectively; C. one-hot encoding: one-hot encoding is performed for the obtained bacteriophage and bacterial SNP features, and the mapping relationship is A={1,0,0,0}, C={0,1,0,0}, G={0,0,1,0}, T={0,0,0,1}; D. merging data: due to the larger difference in encoding length between bacteriophages and bacteria, the bacteriophage encoding should be replicated horizontally multiple times and zeroized to achieve the consistency of encoding dimensions between bacteriophage and bacteria and finally, one-hot encodings of bacteriophages and bacteria obtained above are merged to obtain the final one-dimensional eigenvector. Since the difference in genome size between bacteria and bacteriophages is nearly 10 times, eigenvector of bacteriophage sequence is entirely repeated horizontally multiple times to balance the size difference to avoid the large difference in the eigenvector size. The bacterial eigenvector and replicated bacteriophage vector are merged by row to characterize the location information on the genome of bacteria and bacteriophages and then one-hot encoding A={1,0,0,0}, C={0,1,0,0}, G={0,0,1,0}, T={0,0,0,1} are converted into computer-recognizable information constituting a feature dataset.

The eigenvectors of bacteriophages and bacteria can constitute an eigenvector matrix. For example, the matrix of feature dataset can include two parts: the first four rows are the eigenvector of a bacteriophage, and the last four rows are the eigenvector of a bacterium, which are encoded by onehot (4 bits in column) respectively to represent base type A, T, C and G in gene sequences. The following matrix is a specific example thereof with a dimension of 8, indicating onehot encoding of bacteriophages and bacteria, and 120180 is the number of remaining loci after bacterial gene sequence is aligned and filtered. Due to the shorter sequence of bacteriophage, bacteriophage feature after alignment and filtration should be repeated horizontally and zeroized to make it consistent with the bacterium in the dimension. Finally, eigenvectors of bacteriophage and bacterium are spliced to form the following eigenvector:

$${[ \begin{matrix}1 & 0 & 0 & … & 0 & 0 & 0 \\ 0 & 0 & 0 & … & 0 & 0 & 0 \\ 0 & 1 & 1 & … & 0 & 0 & 0 \\ 0 & 0 & 0 & … & 0 & 0 & 0 \\ 1 & 1 & 0 & … & 0 & 0 & 0 \\ 0 & 0 & 0 & … & 0 & 1 & 1 \\ 0 & 0 & 0 & … & 1 & 0 & 0 \\ 0 & 0 & 1 & … & 0 & 0 & 0\end{matrix} ]}_{8 \times 120180} .$$

For ease of understanding, the above eigenvector matrix can be converted as raw features (the first raw is raw feature of bacteriophage and the second row is raw feature of bacterium), i.e.:

$${[ \begin{matrix}A & C & C & … & 0 & 0 & 0 \\ A & A & G & … & C & T & T\end{matrix} ]}_{2 \times 120180} .$$

### 2. Model Construction and Training Method

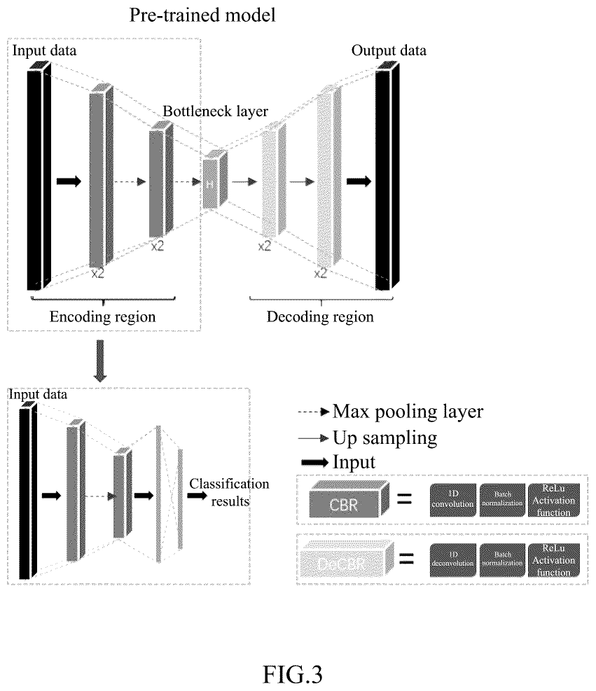

The present invention is based on the neural network framework, and the principle is to construct an End-to-End model, that is, to make prediction based on input data directly, which reduces the intermediate steps. FIG. 3 is a schematic diagram of model framework of the present invention. The model includes a pre-trained model (top) and a master model (bottom) and the main function of the model is to predict whether there is infection relationship between bacteriophages and bacteria based on DNA data. The pre-trained model adopts a convolutional autoencoder model with 1D convolution, and the master model directly adopts the Encoder portion (encoding region) of the pre-trained model plus two layers of full connection. The training data of the two models are the same. The training method of the model is as follows: firstly, the pre-training model is trained; secondly, weights of the Encoder portion in the optimal pre-trained model are applied in the master model as the initial weights for training the master model, and then the model is fine-tuned to complete the model training.

According to the overall framework design of the model as shown in FIG. 3, the model design adopts a two-stage training method and a single-stage prediction method. The model includes a pre-trained model and a master model. The pre-trained model is mainly responsible for helping the weight initialization for the master model, which can effectively avoid problems of overfitting and long convergence time of the master model. FIG. 3 shows the internal design of the deep neural network, where the top half represents the pre-trained model, including two parts, i.e., encoding region and decoding region, both of which are connected by the bottleneck layer. The encoding region and decoding region contain two layers, i.e., CBR module and DeCBR module respectively. The pre-trained model is followed by the master model (the bottom part of FIG. 3), which retains encoding region of the former part, and is followed by two fully connected layers as the classification layer to output the classification results. In FIG. 3, max pooling layer takes the max feature point in the neighborhood of each eigenmatrix; up sampling means that the eigenmatrix is interpolated by optional bilinear interpolation, which aims to gradually restore data with dimensionality reduction to the size of raw data; input indicates the direction of data flow; CBR module (Convolution+Batch Normalization+Relu) includes 1-dimension (1D) convolution layer (convolution operation of 1D convolutional kernel on input data is a conventional feature extraction method for 1D sequence data), batch normalization (the main role of normalization on each batch of training data is to avoid internal covariate shift of each layer in the network when the parameters change) and rectified linear unit (ReLU) (also known as modified linear unit) activation function, which can solve the gradient explosion/gradient vanishment caused by increasing depth of the model. DeCBR (De-Convolution+Batch Normalization+Relu) is almost the same as the CBR module, except that the 1D convolution layer becomes a 1D deconvolution layer, where deconvolution is also known as transposed convolution. Deconvolution is actually the inverse process of convolution, which is a decoding process.

Specifically, in the first stage, the pre-trained model is trained. The eigenvector matrix obtained in step 1 is input into the encoder and the output of the pre-trained model is a set of weight parameters of the optimal model, which is as the basic parameters for the master model. On this basis, training of the master model is fine-tuned to accelerate convergence and reduce oscillation. As shown in FIG. 3, the pre-trained model adopts a standard symmetric autoencoder model, including three parts, i.e., encoder, bottleneck and decoder. The encoder can encode input data into more abstract features. The bottleneck layer can be as the feature data characteristic of input data, then decoder outputs data of the same size as input data, and the model can estimate the learning ability of the model by comparing the similarity between output data and input data. The model architecture of the pre-trained model is shown in Table 1 below, where 1-14 layers are the encoder and 16-28 layers are the decoder:

TABLE 1
1-14 layers are the encoder and 16-28 layers are the decoder.

| Layer | Output Shape | Param # |
|---|---|---|
| Input | [−1, 8, 120975] | — |
| ConvId-1 | [−1, 16, 60473] | 4,112 |
| BatchNormId-2 | [−1, 16, 60473] | 32 |
| ReLU-3 | [−1, 16, 60473] | 0 |
| ConvId-4 | [−1, 32, 30221] | 16,416 |
| BatchNormId-5 | [−1, 32, 30221] | 64 |
| ReLU-6 | [−1, 32, 30221] | 0 |
| MaxPoolId-7 | [−1, 32, 15110] | 0 |
| ConvId-8 | [−1, 16, 7540] | 16,400 |
| BatchNormId-9 | [−1, 16, 7540] | 32 |
| ReLU-10 | [−1, 16, 7540] | 0 |
| ConvId-11 | [−1, 8, 3755] | 4,104 |
| BatchNormId-12 | [−1, 8, 3755] | 16 |
| ReLU-13 | [−1, 8, 3755] | 0 |
| MaxPoolId-14 | [−1, 8, 1877] | 0 |
| Epsample-15 | [−1, 8, 3754] | 0 |
| ConyTransposeId-16 | [−1, 16, 7540] | 4,112 |
| BatchNormId-17 | [−1, 16, 7640] | 32 |
| ReLU-18 | [−1, 16, 7540] | 0 |
| ConyTransposeId-19 | [−1, 32, 15110] | 16,416 |
| BatchNormId-20 | [−1, 32, 15110] | 64 |
| ReLU-21 | [−1, 32, 15110] | 0 |
| Upsample-22 | [−1, 32, 30220] | 0 |
| ConyTransposeId-23 | [−1, 16, 60472] | 16,400 |
| BatchNormId-24 | [−1, 16, 60472] | 32 |
| ReLU-25 | [−1, 16, 60472] | 0 |
| ConyTransposeId-26 | [−1, 8, 120976] | 4,104 |
| BatchNormId-27 | [−1, 8, 120976] | 16 |
| ReLU-28 | [−1, 8, 120976] | 0 |
| Output | [−1, 8, 120976] | — |

The loss function of the pre-trained model adopts Mean-Square Error (MSE), and the optimizer adopts Stochastic Gradient Descent (SGD) algorithm with a learning rate of 0.1. The model is trained until it no longer converges.

$$MSE = \frac{1}{n} {\sum }_{i}^{n} {( {\hat{y}}_{i} - {y}_{i} )}^{2}$$

wherein, yᵢ is the true label, ŷᵢ is the predicted label, and n is the number of samples.

In the second stage, the master model is trained. The eigenvector matrix obtained in step 1 is input into the master model, and the output is the predicted infection results, including predicted infection relationship, predicted infection score and predicted infection possibility. As shown in FIG. 3, backbone of the master model directly follows the encoder architecture in the pre-trained model, which is finally connected with two fully connected layers as the classification layer. During the training process, initial weights are the weights of the optimal model in the pre-trained model. The loss function adopts Cross Entropy Error Function and the classifier adopts Softmax function. That is, for each sample, probability that it belongs to class i is:

$${y}_{i} = \frac{{e}^{{a}_{i}}}{{\sum }_{k = 1}^{c} {e}^{{a}_{k}}} \qquad ∀ i ∈ 1 … C$$

wherein e, as a mathematical constant, is the base of the natural logarithm function, aᵢ represents the output of the i^{th} neuron in the output layer of neural network, and aₖ is the same. C is the number of neurons in the output layer, or the number of classes.

The above formula can guarantee Σᵢ₌₁^{C}yᵢ=1, that is, the sum of probability of each class is 1.

The loss function can be written as:

$$L = - {\sum }_{k = 1}^{n} {\sum }_{i = 1}^{C} {t}_{ki} log ( {y}_{ki} )$$

wherein tₖᵢ, is the probability that sample k belongs to class i, and yₖᵢ is the probability that the model predicts that sample k belongs to class i. C is the number of classes.

The optimizer adopted in the training is Stochastic gradient descen (SGD), the fine-tuning learning rate is 0.01, and Early Stopping module is added, which can effectively resist overfitting.

### 3. Model Prediction and Evaluation

i valid models are obtained in the clades of evolutionary tree, and the prediction of new samples is performed as the following workflow:
1) According to the above, a master model is constructed for each phylogenetic clade, and phylogenetic analysis is performed on genome sequence of the sample to be tested and reference genome of each model X_refᵢ˜{P_refⁱ, B_refⁱ} to determine which clade the sample to be tested belongs to and further determine which master model is used from the integrated model (model combinations).
2) The processing method of genome sequence of new sample is shown in FIG. 2, which is the same as the specific processing method of feature set construction above. Finally, feature data with the same dimension as the training set data, e.g., data acceptable for the model, are obtained.
3) Then, the obtained feature data of new sample is put into the master model determined in the first step for prediction. The master model selection step has been defined within the model, and automatic selection is made based on the phylogenetic results without human operation.

In the present invention, multiple models trained by phylogenetic clades can be combined to form model combinations. In the combined model, phylogenetic analysis is firstly performed for the input bacteriophage in practice to obtain the phylogenetic clade cluster where the bacteriophage is located, and the model of corresponding cluster can be selected to perform the sequence processing and prediction as above.

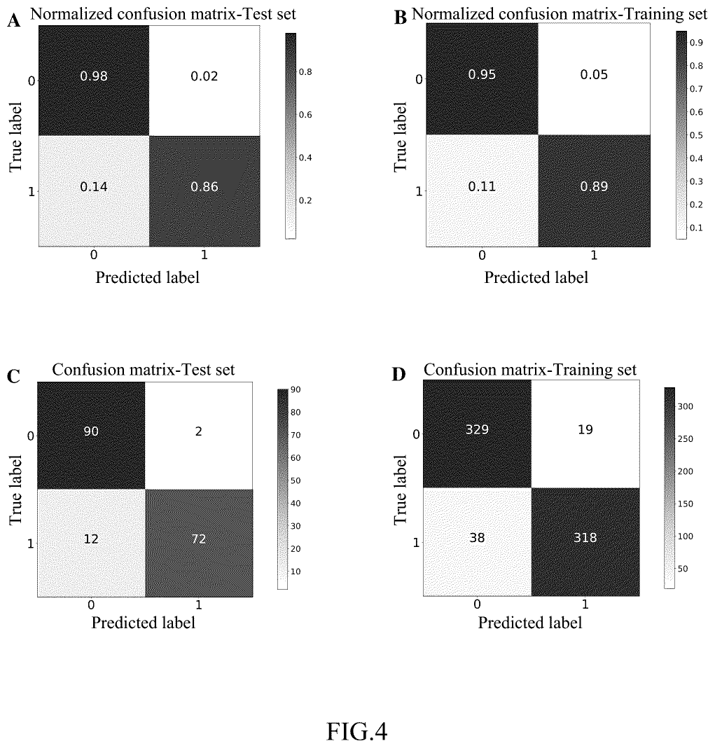

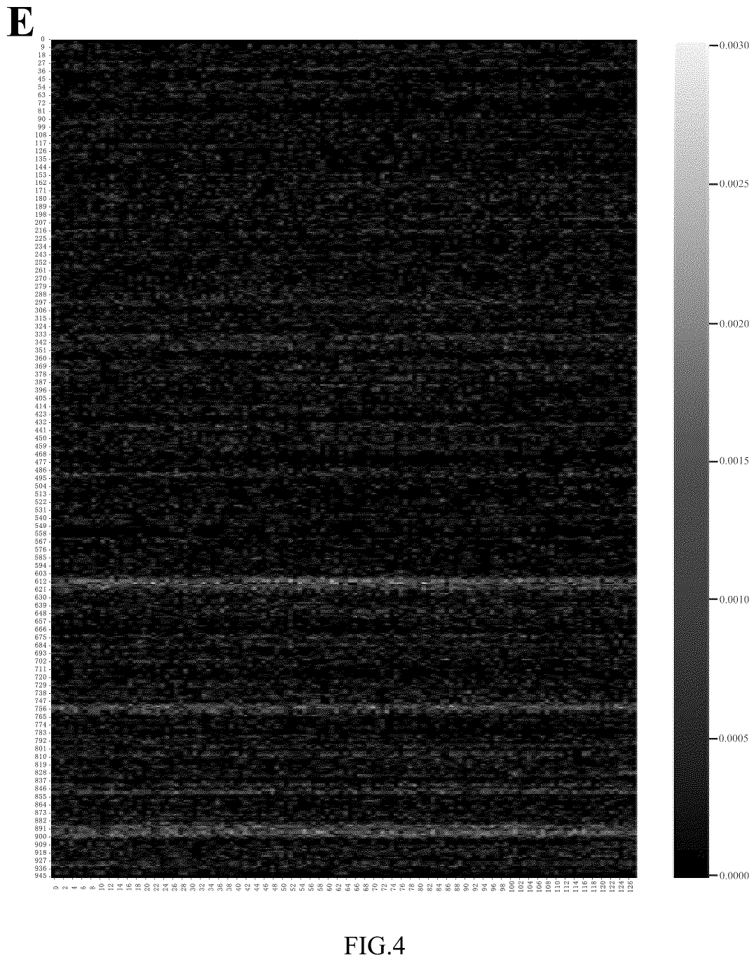

FIG. 4 shows exemplary results of the model. Figures A and B show normalized confusion matrixes of the classification results of test set and training set data of the model, respectively; figures C and D show confusion matrixes of sample numbers of the classification results of test set and training set data of the model, respectively; the confusion matrix summarizes the situation of predicted results of classification model, with each column representing predicted class and each row representing true class of the sample. For example, 90 in the first column of the first row in figure C means that 90 samples that actually belong to negative samples are predicted as negative samples (predicted label is 0), and 2 in the second column of the first row means that 2 samples that actually belong to negative samples are incorrectly predicted as positive samples (predicted label is 1). Similarly, 12 in the first column of the second row means that 12 samples that actually belong to positive samples are incorrectly predicted as negative samples (predicted label is 0), and 72 in the second column of the first row means that 72 samples that actually belong to positive samples are predicted as positive samples (predicted label is 1). Figure A shows the normalization of figure C and the proportion obtained from the normalization of predicted quantities in each class can intuitively and clearly obtain the proportion of correct prediction and wrong prediction. Figures B and D represent results of the mode in the training set, that is, the quality of the training process, and figures A and C illustrate how well the trained model performs in the test set, e.g., data that has never appeared in the model. The results show that the model performs well in the training set, test set and real sample classification with lower probability of false positive and false negative, and the trained model has good generalization ability and can be applied. Figure E represents gradient-weighted class activation mapping calculated by gradient backpropagation of samples, which is formed by fragmenting and merging the input sequence of sample to the length of 128 bp with abscissa representing the length of each segment and ordinate representing the number of segments of the sequence. The brighter portion in the figure indicates that the results of the model are more significant, while the darker portion indicates that the model is less sensitive to it. The results show as follows: 1) based on the above construction principle, 870 groups of bacteriophage-bacterium infection data are divided into the training set (694 groups) and test set (176 groups) at a ratio of 0.2. In the constructed test dataset, the consistency between predicted results and real results is 90% (FIG. 4A, 4B, 4C and 4D); 2) the predicted infection results can be applied in clinical bacteriophage cocktail therapy to provide a choice for the treatment of host bacteria (see example 1, 2, and 3); 3) the predicted significant region of the model (FIG. 4E) can be applied in one of the directions of clinical bacteriophage research and exploration.

In each of phylogenetic cluster clades (FIG. 1), a model for a single cluster can be constructed according to the above method steps, and these models are finally merged into a combined model. Using the computer packaging technology, the training set and test set data, the trained model, the connection calling class of the intermediate process, and the bioinformatics analysis script (such as phylogenetic analysis) are packaged to obtain the final complete version of the software. In practical application, phylogenetic analysis is first performed for the input unknown bacteriophage sequence to obtain the phylogenetic clade cluster where the bacteriophage is located, and the model of corresponding cluster can be selected for sequence processing and prediction as above.

### Example 1: Software Outputs Cocktail Therapeutic Schedule

The bacterial infection condition of the patient is input into the aforementioned packaged software, and the software outputs the corresponding bacteriophage infection score and optimal bacteriophage cocktail formulation therapeutic schedule, as shown in Table 2 below.

TABLE 2
Input: gene sequence (or name) of infective bacterium
Output:

| Infective | | | | |
|---|---|---|---|---|
| bacterial | Infection | Bacteriophage | Infection | |
| genotype | rank | name | score | Confidence |
| BGI- | 1 | PH-KP5996 | 6.80 | 99% |
| 120_KP5493 | 2 | PH-KP6017 | 6.52 | 95% |
| | 3 | PH-KP6031 | 5.48 | 90% |
| | . . . | . . . | . . . | . . . |

Recommendatory cocktail formulation 1: PH-KP5996 (80%) + PH-KP6017 (10%) + PH-KP6031 (10%)
Recommendatory cocktail formulation 2: PH-KP5996 (75%) + PH-KP6017 (15%) + PH-KP6031 (10%)

Table 2 shows clinical application scenario of the final packaged software of the present invention. After gene sequence or name of clinically infective bacterium is input, the name, infection score (predicted value obtained from the model regression fitting prediction) and infection confidence (probability value obtained from the model prediction), etc. of bacteriophage that can infect the bacterium are output within a few minutes. Finally, cocktail formulation recommended by the software will be output and can be used in the clinical therapy.

### Example 2: Software Outputs the Prediction of Bacteriophage Infection Ability

The genomic sequence of bacteriophage with unknown host profile is input into the software, and the software outputs the corresponding bacteriophage infection score, confidence and infection rank to bacteria in the database. Table 3 below shows the application of software to predict the bacteriophage infection score to bacteria in the database.

TABLE 3
Input: gene sequence (or name) of bacteriophage
Output:

| Bacteriophage | Infection | | Infection | |
|---|---|---|---|---|
| genotype | rank | Bacterial name | score | Confidence |
| PH-KP5493 | 1 | BGI-120 KP5493 | 8.80 | 99% |
| | 2 | BGI-135 KP6017 | 3.52 | 95% |
| | 3 | BGI-109 KP6031 | 3.48 | 90% |
| | . . . | . . . | . . . | . . . |

### Example 3: Software Outputs the Prediction of Infection Ability of Bacteriophage-Bacteriophage Pair

The genomic sequences of bacteriophage and bacterium are input into the software, and the software outputs the corresponding bacteriophage-bacterium infection score and confidence. Table 4 below shows the application of software to predict the bacteriophage-bacterium infection score and confidence.

TABLE 4
Input: gene sequence of bacteriophage + bacterial sequence
Output:

| Input | | Predicted infection | Predicted |
|---|---|---|---|
| bacteriophage | Input bacterium | score | confidence |
| PH-KP5493 | BGI-120_KP5493 | 8.80 | 99% |

The method of the present invention covers the positive infection dataset and the negative infection dataset in the real situation of bacteriophages and bacteria, and focuses on analyzing and extracting the sequence features and difference in the level of omics of bacteriophages and bacteria, reaching high accuracy of more than 90% in the classification of bacteriophage infection ability and the infection determination model, which can be applied in the bacteriophage cocktail therapy clinically, and be used to quantitatively predict the infection capacity of bacteriophages that infect bacteria and to recommend available bacteriophage therapeutic formulation.

What is claimed is:

1. A method for constructing a learning model for a bacteriophage-host infection relationship, the method comprising:
training a learning model by using data of bacteriophages and bacterial strains in the training set, the data of bacteriophages and bacterial strains in the training set comprising genomic data and phenotypic data from experimental assay.

2. The method of claim 1, the phenotypic data from experimental assay are the quantitative infection data of bacteriophages and bacterial strains; preferably, the phenotypic data from experimental assay are bacteriophage-bacterium infection score.

3. The method of claim 2, the bacteriophage-bacterium infection score is calculated as follows:
1) circularity is calculated by:
$$circularity = 4 \times π \times area of plaque \div ( perimeter of plaque \times perimeter of plaque ) , \qquad ( 1 )$$
preferably, the plaques with circularity of less than 0.1 are removed;
2) transmissivity of plaque is calculated by:
$$transmissivity of plaque = brightness value of plaque / brightness value of background , \qquad ( 2 )$$
preferably, the plaques with transmissivity of greater than 0.995 are removed;
3) the infection score is calculated by:
$$infection fraction = circularity + 20 \times ( 1 - transmissivity of plaque ) . \qquad ( 3 )$$

4. The method of any one of claims 1-3, the data of bacteriophages and bacterial strains in the training set comprise the data of bacteriophages and bacterial strains without infection relationship.

5. The method of any one of claims 1-3, in the bacteriophages and bacterial strains in the training set, there is the combination of two or more bacteriophages and one bacterial strain; preferably, the two or more bacteriophages are known to infect the one bacterial strain.

6. The method of any one of claims 1-3, the genomic data comprise genomic data of the bacteriophages and bacterial strains, such as genomic sequences; preferably, the genomic data are SNP datasets.

7. The method of claim 6, the SNP datasets comprise the loci where there is difference in the base type on the whole genome or part of genome when individual genome is aligned with reference genome, wherein the loci whose similarity of proportion of base type with the reference genome is greater than a threshold (e.g., 0.9), and invalid loci are deleted.

8. The method of any one of claims 1-3, the data of bacteriophages and bacterial strains in the training set are eigenvector matrix comprising eigenvector of the bacteriophages and eigenvector of the bacteria.

9. The method of any one of claims 1-3, the bacterial strains in the training set are from the same genus; preferably, the bacterial strains in the training set are from the same species; and/or the bacteriophages in the training set are the bacteriophages from the same phylogenetic clade cluster.

10. The method of any one of claims 1-3, the reference sequence of the bacteriophages is the ancestral sequence in each clade, and/or the reference sequence of the bacteria is the reference genome sequence of the bacteria recorded in the NCBI database.

11. The method of any one of claims 1-3, one or more bacterial strains in the same species are infected by one or more bacteriophages in the phylogenetic clade cluster.

12. The method of claim 9 or 11, the phylogenetic clade cluster is divided based on genomic data of the bacteriophages; preferably, the phylogenetic clade cluster is divided by cluster analysis.

13. The method of claim 8, data of the bacteriophages and bacterial strains are merged to form an eigenvector matrix as the input of the learning model; for example, the eigenvector of the bacteriophages is repeated multiple times until its dimension is consistent with that of the eigenvector of the bacterial strains, and the repeated eigenvector of the bacteriophages and the eigenvector of the bacterial strains constitute the eigenvector matrix.

14. The method of any one of claims 1-3, the learning model is based on neural network framework; preferably, the learning model comprises a pre-trained model and a master model; preferably, the pre-trained model adopts a standard symmetric autoencoder model, comprising an encoder, a bottleneck layer and a decoder; preferably, the master model adopts the encoder portion in the pre-trained model and two layers of full connection.

15. The method of any one of claims 1-3, the models trained by multiple different phylogenetic clades are combined to form model combinations, and the model combinations constitute the learning model.

16. A trained learning model obtained by the method of any one of claims 1-15.

17. A method for predicting bacteriophages that are capable of infecting a bacterium, the method comprising:
the genomic data of the bacterium to be tested and each of candidate bacteriophages are input into the trained learning model of claim 16 to predict the bacteriophages or bacteriophage combinations capable of infecting the bacterium to be tested.

18. The method of claim 17, the candidate bacteriophages comprise the bacteriophages used to train the learning model.

19. A method for predicting bacteria infected by a bacteriophage, the method comprising:
the genomic data of the bacteriophage to be tested and each of candidate bacteria are input into the trained learning model of claim 16 to predict the bacteria that the bacteriophage to be tested are capable of infecting.

20. The method of claim 19, the candidate bacteria comprise the bacteria used to train the learning model.

21. A method for predicting the paired infection relationship between bacteria and bacteriophages, the method comprising:
the genomic data of the bacteriophages and bacteria to be tested are pairwise input into the trained learning model of claim 16 to predict whether there is infection relationship between the bacteriophages to be tested and the bacteria to be tested and the infection of the bacteriophages to be tested to the bacteria to be tested.

22. A computer execution medium comprising a computer program, wherein the computer program is used to execute the method steps of any one of claims 17-21.
